SEQUENCE LISTING (18 sequences)

SEQ ID NO: 1 - protein, 758 aa, Homo sapiens
MRKQGVSSKRLQSSGRSQSKGRRGASLAREPEVEEEMEKSALGGGKLPRGSWRSSPGRIQSLKERKGLELEVVAKTFLLGPFQFVRNSLAQLREKVQELQARRFSSRTTLGIAVFVAILHWLHLVTLFENDRHFSHLSSLEREMTFRTEMGLYYSYFKTIIEAPSFLEGLWMIMNDRLTEYPLIINAIKRFHLYPEVIIASWYCTFMGIMNLFGLETKTCWNVTRIEPLNEVQSCEGLGDPACFYVGVIFILNGLMMGLFFMYGAYLSGTQLGGLITVLCFFFNHGEATRVMWTPPLRESFSYPFLVLQMCILTLILRTSSNDRRPFIALCLSNVAFMLPWQFAQFILFTQIASLFPMYVVGYIEPSKFQKIIYMNMISVTLSFILMFGNSMYLSSYYSSSLLMTWAIILKRNEIQKLGVSKLNFWLIQGSAWWCGTIILKFLTSKILGVSDHIRLSDLIAARILRYTDFDTLIYTCAPEFDFMEKATPLRYTKTLLLPVVMVITCFIFKKTVRDISYVLATNIYLRKQLLEHSELAFHTLQLLVFTALAILIMRLKMFLTPHMCVMASLICSRQLFGWLFRRVRFEKVIFGILTVMSIQGYANLRNQWSIIGEFNNLPQEELLQWIKYSTTSDAVFAGAMPTMASIKLSTLHPIVNHPHYEDADLRARTKIVYSTYSRKSAKEVRDKLLELHVNYYVLEEAWCVVRTKPGCSMLEIWDVEDPSNAANPPLCSVLLEDARPYFTTVFQNSVYRVLKVN

SEQ ID NO: 2 - DNA, 2277 bp, Homo sapiens
atgagaaaacaaggagtaagctcaaagcggctgcaatcttccggccgcagccagtctaaggggcggcgcggggcctccctcgcccgggagccggaggtagaggaggagatggaaaagtcggccctaggcggcgggaaactgccaaggggctcctggaggtcctccccggggaggatccaaagtctgaaagagcgaaaaggcttggagctagaggtggtggccaagacctttcttctcggccccttccagttcgtccgtaattccctggcgcagctccgggaaaaggtgcaggaactgcaggcgcggcggttctccagcagaaccactctcggcatcgctgtctttgtggcaattttacattggttacatttagtaacactttttgaaaatgatcgtcatttctctcacctctcatctttggaacgggagatgacttttcgcactgaaatgggactttattattcatacttcaagaccattattgaagcaccttcgtttttggaaggactgtggatgattatgaatgacaggcttactgaatatcctcttataattaatgcaataaaacgcttccatctttatccagaggtaatcatagcctcctggtattgcacattcatgggaataatgaatttatttggactagaaactaagacctgctggaatgtcaccagaatagaacctcttaatgaagttcaaagctgtgaaggattgggagatcctgcttgcttttatgttggtgtaatctttattttaaatggactaatgatgggattgttcttcatgtatggagcatacctgagtgggactcaactgggaggtcttattacagtactgtgcttctttttcaaccatggagaggccacccgtgtgatgtggacaccacctctccgtgaaagtttttcctatcctttccttgtacttcagatgtgtattttaactttgattctcaggacctcaagcaatgatagaaggcccttcattgcactctgtctttccaatgttgcttttatgcttccctggcaatttgctcagtttatactttttacacagatagcatcattatttcccatgtatgttgtgggatacattgaaccaagcaaatttcagaagatcatttatatgaacatgatttcagttacccttagtttcattttgatgtttggaaattcaatgtacttatcttcttattattcttcatctttgttaatgacgtgggcaataattctaaagagaaatgaaattcaaaaactgggagtatctaaactcaacttttggctaattcaaggtagtgcctggtggtgtggaacaatcattttgaaatttctgacatctaaaatcttaggcgtttcagaccacattcgcctgagtgatcttatagcagccagaatcttaaggtatacagattttgatactttaatatatacctgtgctcccgaatttgacttcatggaaaaagcgactccgctgagatacacaaagacattattgcttccagttgttatggtgattacatgttttatctttaaaaagactgttcgtgatatttcatatgttttagctacaaacatttatctaagaaaacagctccttgaacacagtgagctggcttttcacacattgcagttgttagtgtttactgcccttgccattttaattatgaggctaaagatgtttttgacaccgcacatgtgtgttatggcttccttgatatgctctcgacagctctttggctggctttttcgcagagttcgttttgagaaggttatctttggcattttaacagtgatgtcaatacaaggttatgcaaacctccgtaatcaatggagcataataggagaatttaataatttgcctcaggaagaacttttacagtggatcaaatacagtaccacatcagatgctgtctttgcaggtgccatgcctacaatggcaagcatcaagctgtctacacttcatcccattgtgaatcatccacattacgaagatgcagacttgagggctcggacaaaaatagtttattctacatatagtcgaaaatctgccaaagaagtaagagataaattgttggagttacatgtgaattattatgttttagaagaggcatggtgtgttgtgagaactaagcctggttgcagtatgcttgaaatctgggatgtggaagacccttccaatgcagctaaccctcccttatgtagcgtcctgctcgaagacgccaggccttacttcaccacagtatttcagaatagtgtgtacagagtattaaaggttaactga

SEQ ID NO: 3 - DNA, 20 bp, Artificial Sequence
  Primer 3′DPY19L2
ccctcaagcaattctctttg

SEQ ID NO: 4 - DNA, 19 bp, Artificial Sequence
  Primer 3′DPY19L2
gatttgatttggggcctaa

SEQ ID NO: 5 - DNA, 20 bp, Artificial Sequence
  Primer 3′DPY19L2
gattaaagttctggggtgag

SEQ ID NO: 6 - DNA, 20 bp, Artificial Sequence
  Primer 3′DPY19L2
gcagcctattacttccgata

SEQ ID NO: 7 - DNA, 22 bp, Artificial Sequence
  Primer exon 22 DPY19L2
gtgtctgttattaaagcttgtg

SEQ ID NO: 8 - DNA, 23 bp, Artificial Sequence
  Primer exon 22 DPY19L2
attgtctctagacagcaatacat

SEQ ID NO: 9 - DNA, 20 bp, Artificial Sequence
  Primer exon 11 DPY19L2
aacctcctcaagtgacttag

SEQ ID NO: 10 - DNA, 16 bp, Artificial Sequence
  Primer exon 11 DPY19L2
ttggccaagagtcatt

SEQ ID NO: 11 - DNA, 23 bp, Artificial Sequence
  Primer exon 1 DPY19L2
ggccaacttctttctactcggac

SEQ ID NO: 12 - DNA, 23 bp, Artificial Sequence
  Primer exon 1 DPY19L2
gacccagctccaccatactcctt

SEQ ID NO: 13 - DNA, 25 bp, Artificial Sequence
  Primer 5′DPY19L2
ctactattgtctcaaccaaatatgt

SEQ ID NO: 14 - DNA, 22 bp, Artificial Sequence
  Primer 5′DPY19L2
ggaattcaagactagcctagaa

SEQ ID NO: 15 - DNA, 22 bp, Artificial Sequence
  Primer 5′DPY19L2
ggcatggaagttaccacaagaa

SEQ ID NO: 16 - DNA, 20 bp, Artificial Sequence
  Primer 5′DPY19L2
gttacaccactggccgtctc

SEQ ID NO: 17 - DNA, 27 bp, Artificial Sequence
  Primer DPY19L2
aaatctagacacagtcacagcatcatc

SEQ ID NO: 18 - DNA, 27 bp, Artificial Sequence
  Primer DPY19L2
agaggcttaataggtgaaagaaagaga

(19) United States
(12) Patent Application Publication
(10) Pub. No.: US 2012/0283126 A1
(43) Pub. Date: Nov. 8, 2012

(54) HUMAN FERTILITY TEST USING DPY19L2

(75) Inventors: Pierre Ray, Saint Ismier (FR); Christophe Arnoult, Saint Etienne de Crossey (FR)

(73) Assignee: UNIVERSITE JOSEPH FOURIER (GRENOBLE 1), Grenoble (FR)

(21) Appl. No.: 13/509,713

(22) PCT Filed: Nov. 10, 2010
(86) PCT No.: PCT/FR2010/052416

§ 371 Date: Jul. 24, 2012

(30) Foreign Application Priority Data
Nov. 12, 2009 (FR) 0957963

(51) Int. Cl. C12Q 1/68; C07H 21/04; C07H 21/02; C07H 21/00; C07K 16/18; C07K 14/47; C07K 17/00; C12N 15/85; C40B 30/04

(52) U.S. Cl. 506/9; 435/6.12; 435/320.1; 536/24.5; 530/387.9; 530/350; 435/6.1; 530/402; 536/23.5

(57) ABSTRACT

The present patent application concerns a method for diagnosing infertility related to the DPY19L2 protein, in a male human subject, and a method for treating said subject with a view to restoring fertility. The present invention also concerns the use of molecules inhibiting the action of the DPY19L2 polypeptide in a wild-type carrier male subject, for example of anti-DPY19L2 interfering nucleic acid type selectively inhibiting the expression of the DPY19L2 polypeptide.

### TECHNICAL FIELD

The present patent application concerns a method for diagnosing infertility, more precisely infertility associated with the DPY19L2 protein in a male human subject, and a method for treating such subjects with a view to restoring fertility. The present invention also concerns the use of molecules inhibiting the action of the DPY19L2 polypeptide in a wild-type carrier male subject, for example molecules such as anti-DPY19L2 interfering nucleic acids selectively inhibiting the expression of the DPY19L2 polypeptide.

### BRIEF DISCUSSION OF RELATED ART

At the present time, the only oral contraceptives available are hormonal female contraceptives of oestro-progesterone type.

Spermatogenesis is a highly specialized function which represents a good target for the development of alternative contraceptive methods. Up until now very few genes have been able to be related to spermatogenesis disorders. It was shown in the 1970's that micro-deletions of chromosome Y led to oligozoospermia (reduction in the number of spermatozoa) or azoospermia (lack of spermatozoa). The complexity of the region which is formed of repeat sequences has not allowed a clear role to be determined for the genes located in this region. Since this discovery and up until 2003 no human gene has been able to be formally associated with spermatogenesis disorders in man.

In 2003, Xu Min and colleagues identified a new gene involved in spermatogenesis: SPATA16/NYD-SP12 (Xu Min et al., 2003). This gene, when it has mutated, gives a rare infertility phenotype: globozoospermia (Dam et al. 2007; WO2009/013405).

Also, in 2007, Dieterich et al showed that the absence of the functional protein Aurora Kinase C (AURKC) through the c.144delC mutation led to primary infertility via the production of macrocephalic spermatozoa (Dieterich et al., 2007).

The inventors of the present invention have evidenced a new gene involved in spermatogenesis: DPY19L2. Complete homozygous deletion of the gene leads to a globozoospermia phenotype, a phenotype that is fully incompatible with natural reproduction.

The DPY-19 gene has been described as being involved in the polarity of neuroblasts and their migration along the antero-posterior axis in the yeast C. elegans (Honigberg et al., 2000). Nonetheless, up until the present time, DPY19L2 has never been described as being involved in spermatogenesis and human infertility.

The inventors have shown that the DPY19L2 protein is necessary for the formation of the acrosome and elongation of spermatozoa heads. If the DPY19L2 protein is lacking, the spermatozoa are devoid of an acrosome and are unable to enter into the ovule for fertilization thereof. Therefore a person carrying the mutation, leading to total absence of the DPY19L2 protein or to a mutant DPY19L2 protein, suffers from infertility related to the absence of the DPY19L2 gene. In addition, patients having said phenotype do not have any health problems other than their infertility, which makes DPY19L2 a target of choice for the development of a male contraceptive.

### BRIEF SUMMARY

The present invention concerns a DPY19L2 polypeptide characterized in that it comprises or consists of a polypeptide chosen from among:
the polypeptide of sequence SEQ ID No. 1;
a polypeptide having a function in the formation of the acrosome of spermatozoa and a sequence which has at least 80% identity to SEQ ID No. 1.

The present invention also concerns a polynucleotide encoding a DPY19L2 polypeptide such as defined above. According to one embodiment, this polynucleotide is characterized in that it has the sequence SEQ ID No. 2 or the complementary sequence of SEQ ID No. 2.

The invention also concerns a polypeptide encoded by the nucleotide sequence lying on human chromosome 12 between nucleotide 63,952,693 and nucleotide 64,062,354, of NCBI accession number: NC_{—}000012.11, between the genes AVPR1A and TMEM5 (FIG. 3).

According to one embodiment of the invention, the polypeptide is used in a male human subject with a view to restoring functional spermatogenesis.

The invention also concerns an antibody specific to the DPY19L2 polypeptide of the invention.

The invention also concerns a diagnostic or prognostic method for infertility of globozoospermia type related to DPY19L2 in a male human subject, the DPY19L2 gene being located at 12q14.2, the said method comprising a step consisting of detecting, in a biological sample of the said subject, the presence of absence of a polynucleotide lying on chromosome 12 between nucleotide 63,952,693 and nucleotide 64,062,354, of NCBI accession number: NC_{—}000012.11.

The invention also concerns an expression cassette comprising a functional promoter, a polynucleotide according to the invention and a terminator sequence; and a vector comprising the polynucleotide according to the invention and/or an expression cassette according to the invention, for example for use thereof to treat a male human subject having DPY19L2-related infertility.

The invention also concerns a molecule inhibiting the expression or action of the DPY19L2 polypeptide according to the invention, in wild-type carrier males. According to one embodiment of the present invention, it is an anti-DPY19L2 interfering nucleic acid of DNA or RNA type selectively inhibiting the expression of the DPY19L2 polypeptide in wild-type carrier males. According to one embodiment, this molecule is used as contraceptive means in wild-type carrier males.

Finally the invention concerns a solid substrate of chip type on which the polypeptide of the invention or the polynucleotide of the invention is fixed.

More specifically, the subjects of the invention are the following:
A method for diagnosing infertility in a male human subject, the said method comprising a step consisting of detecting, in a biological sample of the said individual, the presence or absence of a polynucleotide on chromosome 12, between the nucleotide 63,952,693 and the nucleotide 64,062,354, of NCBI accession number: NC_{—}000012.11; according to this method, the homozygous or heterozygous deletion of the said polynucleotide is detected; in the method of the present invention, the said polynucleotide is the DPY19L2 gene.
A method for diagnosing infertility in a male human subject according to which, in a biological sample of the said individual, no nucleotide sequence identical to SEQ ID NO: 2 is detected, or a nucleotide sequence is detected differing from SEQ ID NO: 2 but having at least 95% identity to SEQ ID NO: 2; for this method of the invention, one preferred nucleotide sequence is a nucleotide sequence differing from SEQ ID NO: 2 but having at least 95% identity to SEQ ID NO: 2, and which does encode the DPY19L2 protein.

Any one of the methods of the invention may comprise a prior amplification step by means of primers chosen from among the pairs SEQ ID NOS: 3 and 4, SEQ ID NOS: 5 and 6, SEQ ID NOS: 7 and 8, SEQ ID NOS: 9 and 10, SEQ ID NOS: 11 and 12, SEQ ID NOS: 13 and 14, SEQ ID NOS: 15 and 16 and SEQ ID NOS: 17 and 18. Negative amplification with at least one of the pairs SEQ ID NOS: 7 and 8, SEQ ID NOS: 9 and 10, SEQ ID NOS: 11 and 12, and preferably with at least two of these pairs, and better still with the three pairs, is characteristic of homozygous absence of the DPY19L2 gene. Positive amplification with at least one of the pairs SEQ ID NOS: 7 and 8, SEQ ID NOS: 9 and 10, SEQ ID NOS: 11 and 12, preferably with at least two of these pairs, and better still with the three pairs, and positive amplification with the pair SEQ ID NOS: 17 and 18 is characteristic of heterozygous absence of the DPY19L2 gene.

A biological sample is advantageously chosen from among the leukocytes of peripheral blood and saliva.

A method of the invention such as described above is advantageously coupled with histological analysis of the individual's spermatozoa.

### BRIEF DESCRIPTION OF THE FIGURES

FIG. 1: confocal microscopy images (A, D, G), electron microscope images (B, E, H) and microscopy scan (C, F, I) of control spermatozoa (A, B, C), globozoocephalic spermatozoa without acrosome (D, E, F) globozoocephalic spermatozoa having an atrophied or ill-placed acrosome (G, H, I).

FIG. 2: PCR amplification of 7 loci of the DPY19L2 gene [bands a) to g)] in 10 patients suffering from globozoospermia (P1-P9 and P6f) and in 4 fertile patients (T1-T4); the loci located in the gene (c, d and e) are not amplified, nor are the loci b and f; deletion is delimited by the loci a and g which give similar amplification between patients and controls.

FIG. 3: schematic illustration of the DPY19L2 gene; the loci a) and b) are approximately located at 9 and 25 kb from the 3′ end of DPY19L2; the loci c), d) and e) respectively show the position of the exons 22, 11 and 1 of the DPY19L2 gene; the regions 1 and 2 represent duplicated sequences of 28 kb having 98% homology located either side of the DPY19L2 gene and suggest that the deletions result from a phenomenon of homologous recombination. The scheme at the bottom of the page shows the deletion present in mutant patients. The h primers allow amplification, solely in deleted patients, of a fragment of 1450 nt which comprises the breakpoints of the deletion.

FIG. 4: PCR amplification using h primers in 8 patients (P1, P2, P4, P5, P6, P7, P8 and P9) and 7 controls (T1 to T7).

FIG. 5: CGH analysis (Comparative Genomic Hybridization) of patient P1

### DETAILED DESCRIPTION

### Description of the Sequences

SEQ ID No. 1: Peptide sequence of DPY19L2 (NCBI accession number: NC_{—}000012.11)

SEQ ID No. 2: cDNA sequence of DPY19L2 (NCBI accession number: NC_{—}000012.11)

SEQ ID Nos. 3-18: Primers for cloning

### Polypeptides

The subject of the invention concerns DPY19L2 polypeptides having a function in the formation of spermatozoa acrosomes and in the elongation of the head of spermatozoa.

As explained above, the inventors have shown that the DPY19L2 protein is necessary for the formation of the acrosome and elongation of the head of spermatozoa. If the DPY19L2 protein is absent, the spermatozoa are devoid of an acrosome and cannot enter into the ovule for fertilization thereof. Therefore a person carrying the mutation, which leads to total absence of the DPY19L2 protein or a mutant DPY19L2 protein, suffers from infertility related to absence of the DPY19L2 gene.

Immunohistochemical experiments have provided confirmation that in patients with a deletion for DPY19L2 the acrosome was either fully absent or atrophied and ill-positioned on the sperm. The inventors have also shown that globozoospermia in some patients is related to the absence of the DPY19L2 protein on chromosome 12 (at 12q14.2; more precisely on chromosome 12 between nucleotide 63,952,693 and nucleotide 64,062,354, of NCBI accession number: NC_{—}000012.11) between the genes AVPR1A and TMEM5 (FIG. 3).

The DPY19L2 polypeptide of the invention is shown in sequence SEQ ID No. 1 of the sequence listing.

Another subject of the invention concerns polypeptides having a function in the formation of spermatozoa acrosomes and having at least 80% identity to the polypeptide of SEQ ID No. 1. A further subject of the invention concerns polypeptides having at least 90%, 95%, 98% and preferably at least 99% of identical amino acids to the polypeptide of SEQ ID No. 1.

By <<identical amino acids>> is meant invariant amino acids between two sequences. These polypeptides may exhibit a deletion, an addition or a substitution of at least one amino acid relative to the polypeptide of SEQ ID No. 1.

A further subject of the invention concerns polypeptides having at least 80%, 90%, 95%, 98% and preferably at least 99% similarity with the polypeptide of SEQ ID No. 1. By <<similarity>> is meant measurement of the resemblance between protein or nucleic sequences. These polypeptides may display a deletion, an addition or a substitution of at least one amino acid relative to the polypeptide of SEQ ID No. 1. The degree of similarity between two sequences, as quantified by a score, is based on sequence conserving percentage identity and/or substitution.

The methods for measuring and identifying the degree of identity or degree of similarity between polypeptides are known to persons skilled in the art. It is possible for example to use Vector NTi 9.1.0, an AlignX alignment programme (Clustal W algorithm) (Invitrogen INFORMAX, http://www.invitrogen.com). Preferably the default parameters are used.

The invention also concerns a polypeptide encoded by the nucleotide sequence on human chromosome 12 between nucleotide 63,952,693 and nucleotide 64,062,354, of NCBI accession number: NC_{—}000012.11 between the genes AVPR1A and TMEM5 (FIG. 3).

The invention also concerns a DPY19L2 polypeptide according to the invention for use in a male human subject with a view to restoring functional spermatogenesis.

### Polynucleotides

The invention also concerns polynucleotides coding for the DPY19L2 polypeptide of the invention. Preferably, these polynucleotides encode the polypeptide of sequence SEQ ID No. 1.

According to the present invention, by 0 polynucleotide>> is meant a single strand nucleotide chain or its complementary possibly being of DNA or RNA type, or a double strand nucleotide chain (DNA). Preferably, the polynucleotides of the invention are of DNA type, in particular double strand DNA. The term <<polynucleotide>> also designates modified polynucleotides. The invention generally relates to polynucleotides encoding the polypeptides of the invention. Owing to degeneration of the genetic code, different polynucleotides may encode one same polypeptide.

The polynucleotides of the present invention are isolated, amplified or purified of their natural environment. Preferably the polynucleotides of the present invention can be prepared using conventional molecular biology techniques such as described by Sambrook et al. (T. Maniatis, E. F. Fritsch, J. Sambrook, Molecular Cloning: A Laboratory Handbook, 1982), or by chemical synthesis.

In one embodiment of the present invention, the polynucleotide coding for the DPY19L2 polypeptide comprises the sequence SEQ ID No. 2 or the complementary sequence of SEQ ID No. 2.

The invention also concerns the nucleotide sequence on human chromosome 12 between nucleotide 63,952,693 and nucleotide 64,062,354 of NCBI accession number: NC_{—}000012.11. This nucleotide sequence lies between the genes AVPR1A and TMEM5 (FIG. 3).

### Diagnosis and Prognosis

The present invention also concerns a diagnosis or prognosis method for DPY19L2-related infertility of globozoospermia type in a male human subject. This method comprises a step which consists of detecting the presence or absence of a polynucleotide on chromosome 12 between nucleotide 63,952,693 and nucleotide 64,062,354 of NCBI accession number: NC_{—}000012.11. This polynucleotide lies between the genes AVPR1A and TMEM5 (FIG. 3).

In other words, the method of the invention comprises a step which consists of detecting the presence of the polynucleotide encoding the DPY19L2 polypeptide of the invention or a fragment thereof, in a biological sample of the said patient. Alternatively the method comprises a step which consists of detecting the absence of the polynucleotide encoding the DPY19L2 polypeptide of the invention. According to another alternative, the method comprises a step which consists of detecting the presence of a mutant DPY19L2 polynucleotide.

The method comprises steps and means for determining whether the DPY19L2 gene of a male human subject has complete deletion of the DPY19L2 gene or a mutant sequence of the gene relative to the sequence of the human wild-type DPY19L2 gene. Carriers of the wild-type DPY19L2 gene show a fertile phenotype compatible with natural reproduction.

With the method of the invention, it is therefore possible to detect the presence of the human wild-type DPY19L2 gene, of the human mutant DPY19L2 gene or absence of the human DPY19L2 gene, in a biological sample of a male individual.

If the individual has complete deletion of the DPY19L2 gene or a mutant sequence of the gene relative to the sequence of the human wild-type DPY19L2 gene, the diagnosis/prognosis of infertility or insufficient fertility for natural reproduction may be made and the solutions intended to remedy the situation can be set in place or anticipated.

If the male individual has a human wild-type DPY19L2 gene, the diagnosis of infertility is negative which means that the individual does not suffer from DPY19L2-related infertility of globozoospermia type. In this case, the practitioner will possibly search for the presence or absence of other diagnostic markers.

By <<mutant DPY19L2 gene>> or <<mutant DPY19L2 polynucleotide>> is meant a nucleotide sequence which derives from the human wild-type DPY19L2 gene via a deletion, addition or substitution of at least one nucleotide so as to encode a mutant DPY19L2 protein. This at least one deletion, addition or substitution (or these deletions, additions and/or substitutions) generally occurs in at least one of the sequences of the gene. For example, it may occur in one or more coding sequences (CDS) of the gene. Alternatively, it may occur in sequences not encoding the gene (in particular sequences acting on splicing or regulation of the expression of the gene, or the sequences which delimit the introns-exons). The DPY19L2 gene comprises 22 exons. One example of a mutant gene according to the invention is a fragment of the human wild-type DPY19L2 gene comprising part but not all of the human wild-type DPY19L2 polynucleotide from which it derives, from which an exon for example is missing relative to the wild-type sequence.

By <<mutant DPY19L2 protein>> or <<mutant DPY19L2 polypeptide>> is meant an amino acid sequence which derives from the human wild-type DPY19L2 protein by a deletion, addition or substitution of at least one amino acid and which on this account has lost its function in the formation of the acrosome of sperm.

Diagnosis is based on a biological sample of the male patient. Detection is carried out using conventional molecular biology techniques known to those skilled in the art, such as those described by Sambrook et al. (T. Maniatis, E. F. Fritsch, J. Sambrook, Molecular Cloning: A Laboratory Handbook, 1982). It is generally necessary to use nucleic acid probes such as described in the examples below, more particularly in Table 1. The sequences of these probes SEQ ID Nos. 3-12 can be found in the sequence listing.

Therefore, according to one embodiment of the present invention, the method of the invention comprises a step consisting of detecting, in a biological sample of a patient, the presence of the polynucleotide encoding the DPY19L2 polypeptide of the invention, the presence of a fragment of the polynucleotide encoding the DPY19L2 polypeptide of the invention, the presence of a mutant polynucleotide having at least one mutation e.g. an addition, a deletion or a substitution of at least one nucleotide relative to the polynucleotide encoding the DPY19L2 polypeptide of the invention, and/or the absence of the polynucleotide encoding the DPY19L2 polypeptide of the invention, using a pair of specific nucleic acid probes. The probes may be specific to the polynucleotide encoding the DPY19L2 polypeptide of the invention, or to a fragment thereof or to the mutant polynucleotide having at least one mutation e.g. an addition, a deletion or a substitution of at least one nucleotide relative to the polynucleotide encoding the DPY19L2 polypeptide of the invention.

A further subject of the invention is a diagnosis reagent characterized in that it is chosen from the group formed by the polynucleotides, polypeptides and antibodies of the invention.

Yet a further subject of the invention is a diagnosis or prognosis method for infertility of DPY19L2-related globozoospermia type in a male individual, characterized in that it comprises the contacting of a biological sample of the said individual with a diagnostic reagent such as defined above e.g. an antibody, and the detection of the formation of an antibody and polypeptide complex present in the biological sample.

### Primers

The present invention also concerns a pair of primers specific to the human wild-type DPY19L2 polynucleotide according to the invention in particular the one represented by sequence SEQ ID No. 1 and allowing its selective amplification under sufficient stringency conditions.

By <<primer>> (or by <<probe>>) is meant a short sequence of oligonucleotides which, hybridized with a matrix of nucleic acids, allows a polymerase to initiate the synthesis of a new DNA strand. The strand produced from the primer is complementary to the strand used as matrix. Repeat of the synthesis by Polymerase Chain Reaction (PCR) allows millions of copies of the sequence of interest to be obtained.

### Expression Cassettes and Vectors

According to one embodiment of the invention, a polynucleotide encoding a polypeptide according to the invention is inserted in an expression cassette using cloning techniques well known to a skilled person. This expression cassette comprises the elements needed for transcription and translation of the sequences coding for the polypeptides of the invention.

Advantageously, the expression cassette comprises elements allowing a polypeptide to be produced by a host cell and the elements needed for regulating this expression.

These expression cassettes, in the direction of transcription, comprise:
a functional promoter;
a polynucleotide according to the invention; and
a terminator sequence.

Any type of promoter sequence can be used in the expression cassettes of the invention. The choice of promoter will notably depend on the host organism chosen for the expression of the gene of interest. Some promoters allow constitutive expression whereas, on the contrary, other promoters are inducible.

The present invention also concerns replication or expression vectors comprising at least one polynucleotide or an expression cassette such as previously described. This vector may in particular be a plasmid, a cosmid, a bacteriophage or a virus in which a polynucleotide or expression cassette of the invention is inserted. The techniques for constructing these vectors and for inserting a polynucleotide according to the invention in these vectors are well known to persons skilled in the art.

The techniques for vector construction, transformation of host organisms and expression of heterologous proteins in these organisms are described in the literature (Ausubel F. M. et al. “Current Protocols in Molecular Biology” Volumes 1 and 2, Greene Publishing Associates and Wiley-Interscience, 1989; T. Maniatis, E. F. Fritsch, J. Sambrook, Molecular Cloning: A Laboratory Handbook, 1982).

### Inhibitor Molecules

The present invention also concerns a molecule inhibiting the expression or the action of the DPY19L2 polypeptide according to the invention in wild-type carrier males. This molecule represents male contraceptive means. The spermatozoa are devoid of an acrosome or have a non-functional acrosome under the effect of treatment with the inhibitor molecule. This is a result of the direct action of the molecule on the DPY19L2 gene, the mRNA derived from transcription of the DPY19L2 gene or the DPY19L2 protein. The molecule of the invention is able to act so as to block the transcription or the translation of the DPY19L2 gene. Alternatively, it can block the action of the protein thereby leading either to an acrosome that is completely absent or to an atrophied acrosome ill-positioned on the sperm.

In one embodiment of the present invention, it is an anti-DPY19L2 interfering nucleic acid of DNA or RNA type which selectively inhibits the expression of the DPY19L2 polypeptide in wild-type carrier males. In particular this may be siRNA or shRNA.

The invention also concerns a molecule according to the invention for use thereof as contraceptive means in wild-type carrier males. Finally, the invention concerns the use of the molecule according to the invention for preparing a contraceptive medicinal product for male individuals that are wild-type carriers.

### Solid Substrate

The polynucleotides and polypeptides of the present invention can be immobilized on a solid substrate.

Solid substrates are known that are adapted for immobilizing polynucleotides, in particular for the fabrication of DNA chips. Numerous varieties of DNA chips exist which differ through the type of substrate used, the type, density and fixing or synthesis mode of nucleotide sequences on the substrate, and reading conditions. These techniques are known to those skilled in the art.

By solid substrate is also meant substrates of microsphere type.

### Antibodies

A further subject of the present invention concerns antibodies characterized in that they are directed against one or more DPY19L2 polypeptides of the present invention.

According to one embodiment of the invention, the said antibodies are chosen from among monoclonal antibodies and polyclonal antibodies.

### EXAMPLES

Globozoospermia is a rare phenotype of primary total male infertility characterized by the production of spermatozoa devoid of an acrosome (OMIM #102530).

The inventors have recently shown that the strategy of homozygosis mapping using screening over the breadth of the genome applied to infertile patients from the same ethnic-geographical background leads to the locating and identification of genes involved in spermatogenesis. This genetic strategy was applied to a cohort of Tunisian patients suffering from complete globozoospermia. The inventors have shown that 8 out of the 10 patients analysed had homozygous deletion for DPY19L2.

### Patients, Material and Method

Patients (or Male Human Subject)

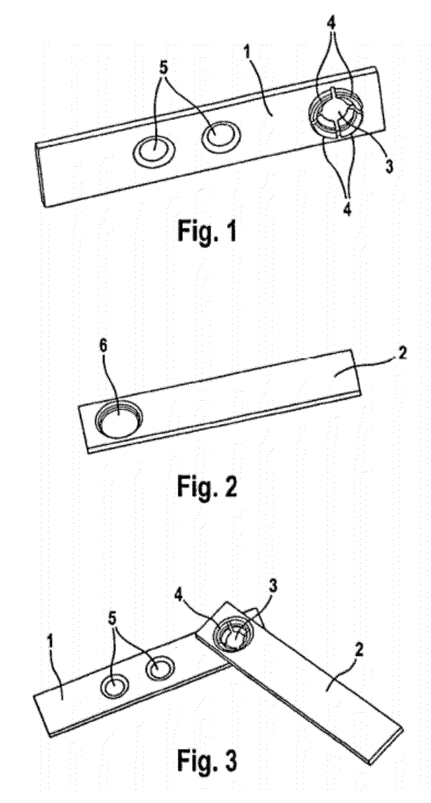

All the individuals had round-headed spermatozoa (FIG. 1). All sperm analyses were carried out at least twice following the recommendations of the World Health Organisation. All the individuals were of normal somatic karyotype.

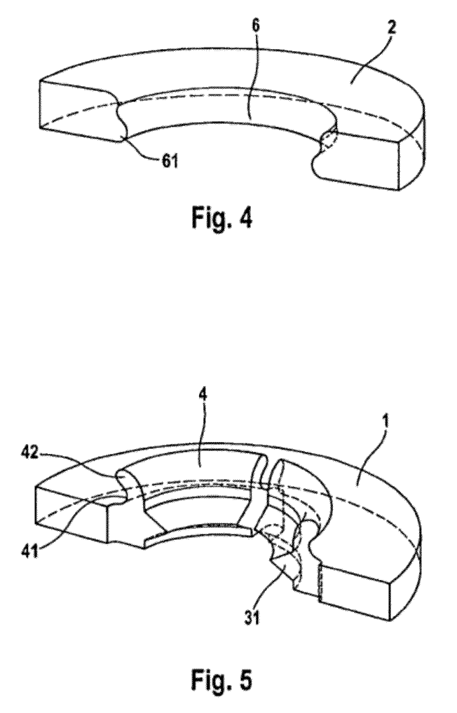

Patients P1 to P6, P8 and P9 (FIG. 2 and FIG. 4) are Tunisians treated in Tunis for infertility, born into the same family e.g. first cousins.

Patient P7 is of Turkish origin living in France. There is no family consanguinity.

Patients P6 and P6f are brothers.

### Molecular Analyses

Genomic DNA was extracted from the leukocytes of peripheral blood using an extraction procedure on guanidium chloride, or from saliva using the Oragene DNA Self-Collection kit (DNAgenotech, Ottawa Canada).

The primers used and the hybridization temperatures are given in Table 1 below:

| | 5′ - 3′ DNA sequences | | Hybridiza- |
|---|---|---|---|
| | of primers sense (F, forward) | | tion Temp |
| Amplified locus | and antisense (R, reverse) | Size (nt) | (° C.) |
| a) 3′DPY19L2 | F: CCCTCAAGCAATTCTCTTTG | 288 | 60 |
| | (SEQ ID No 3) | | |
| | R: GATTTGATTTGGGGCCTAA | | |
| | (SEQ ID No 4) | | |
| b) 3′DPY19L2 | F: GATTAAAGTTCTGGGGTGAG | 444 | 60 |
| | (SEQ ID No 5) | | |
| | R: GCAGCCTATTACTTCCGATA | | |
| | (SEQ ID No 6) | | |
| c) exon 22 | F: GTGTCTGTTATTAAAGCTTGTG | 313 | 57 |
| | (SEQ ID No 7) | | |
| | R: ATTGTCTCTAGACAGCAATACAT | | |
| | (SEQ ID No 8) | | |
| d) exon 11 | F: AACCTCCTCAAGTGACTTAG | 516 | 57 |
| | (SEQ ID No 9) | | |
| | R: TTGGCCAAGAGTCATT | | |
| | (SEQ ID No 10) | | |
| e) exon 1 | F: GGCCAACTTCTTTCTACTCGGAC | 504 | 65 |
| | (SEQ ID No 11) | | |
| | R: GACCCAGCTCCACCATACTCCTT | | |
| | (SEQ ID No 12) | | |
| f) 5′DPY19L2 | F: CTACTATTGTCTCAACCAAATATGT | 610 | 58 |
| | (SEQ ID No 13) | | |
| | R: GGAATTCAAGACTAGCCTAGAA | | |
| | (SEQ ID No 14) | | |
| g) 5′DPY19L2 | F: GGCATGGAAGTTACCACAAGAA | 750 | 65 |
| | (SEQ ID No 15) | | |
| | R: GTTACACCACTGGCCGTCTC | | |
| | (SEQ ID No 16) | | |
| h) DPY19L2 | F: AAATCTAGACACAGTCACAGCATCATC | 1450 | 60 |
| breakpoint | (SEQ ID No 17) | | |
| | R: AGAGGCTTAATAGGTGAAAGAAAGAGA | | |
| | (SEQ ID No 18) | | |

35 PCR amplification cycles were performed using Taq polymerase (Qiagène, Courtaboeuf, France).

Sequencing was carried out using BigDye Terminator v3.1 (Applied Biosystems). Electrophoresis analyses were performed on ABI 3130XL (Applied Biosystems).

### Microarray Analyses

250K Sty1 SNP microarrays (Affymetrix, Inc., Santa Clara, Calif.) were applied to patients P1-P9 following the manufacturer's directions. Analyses were conducted at the IGBMC centre (Strasbourg).

### Electron Microscopy

The spermatozoa were fixed with 2.5% glutaraldehyde in 0.1 M cacodylate buffer pH 7.4 for 2 hours at ambient temperature. The cells were then washed with buffer and post-fixed with 1% osmium tetroxide in the same buffer for 1 hour at 4° C. After extensive washing with water, the cells were fixed with 0.5% uranyl acetate pH 4 overnight at 4° C. The cells were then dehydrated using an alcohol gradient (30%-60%-90%-100%-100%-100%) and infiltrated with a 1:1 mixture of epon/100% alcohol for one hour before being washed several times in fresh epon (Flukka) for three hours. Finally, the cells were centrifuged and immersed in fresh epon and polymerized for 3 days at 60° C. Ultra-thin sections of the cells were prepared using an ultra-microtome (Leica). The sections were post-fixed with 4% uranyl acetate and 1% lead citrate before observation under electron microscope operated at 80 kV (JEOL 1200EX).

### Results

Detailed Phenotype Analyses

The 10 patients described here suffered from complete globozoospermia with 100% round-headed spermatozoa and increased percentage of flagellar defects (40%). All the patients had normal sperm volume (4.6 ml). Sperm concentration was normal in 7 patients (52-126 Million/ml) and patients P5, P7 and P8 had severe oligozoospermia of between 0.02-1.5 M/ml. The mean motility and morbidity in the 7 first patents was 27.5%, a value higher than the mean. The MAI index (Multiple Anomalies Index) was high with a mean of 2.5. Additional analyses were carried out in patient P1. Dual marking of the nucleus and acrosome with TOPRO-3 and Pisum savitum lectins respectively, showed perfectly round nuclei (FIG. 1D, G), without an acrosome (PSA-FITC labelling, not illustrated—FIG. 1D) or with foci of ill-positioned acrosomes on the periphery of the nucleus (PSA-FITC labelling, not illustrated—FIG. 1G). Transmission electron microscopy confirmed the presence of round nuclei (FIG. 1E, H). The photographs show that the contour of the round spermatozoa is not smooth (FIG. 1F, I). The sperm surface appears wrinkled by what could be a plasma membrane that is insufficiently taut or a practically empty acrosome membrane. The irregularity of this outer membrane can be seen in photograph1H.

### Locating of the Candidate Gene

Screening over the breadth of the genome in 9 affected patients was carried out using SNP Affimetrix chips (250K Sty1 SNP arrays). It was sought to identify common homozygous regions. 7 patients shared a homozygous region of between 0.4 and 54 million nucleotides (MB) centred at 12q14.2. 4 candidate genes were present in the homozygous region. An in silico study of the expression profile of these 4 candidate genes evidenced the fact that DPY19L2 is predominantly expressed in the testicles.

### Identification of the Mutation

The primers c, d and e allowed detection of the presence of the homozygous deletion of the DPY19L2 gene. PCR analysis in the deleted patients was negative with these primers. The primers a, f allowed delimiting of the deletion and were used as positive controls for amplification in the patients.

The h primers allowed amplification of the recurrent deletion found in the globozoocephalic patients. This PCR was therefore positive in affected males and negative in non-affected males.

In addition, these primers allowed the screening of individuals carrying a deletion present in the heterozygous state: PCR c, d, e positive and PCR h positive.

PCR amplification of the DPY19L2 exons showed that the gene was deleted in all the patients except P7 and P8, patient P7 being the patient of Turkish origin. The smallest homozygous region used to locate (and identify) the candidate gene was the region in patient P7. Logically, patient P6f, recruited at a later stage, also showed homozygous deletion of the DPY19L2 gene. Subsequent amplifications on each side of the gene allowed the determination of two breakpoints of 15 KB located on each side of DPY19L2 (FIG. 3). The deletion approximately includes a region of 200 KB housing only the DPY19L2 gene.

PCR with primers f allowed amplification of the deleted region in the patients and hence led to diagnosis of heterozygosis and homozygosis.

PCR amplification of two exons (1 and 11) was performed in 100 North African fertile control patients. None was homozygous deleted for DPY19L2. Also, the epidemiological data given in Table 2 below (public data obtained from http://cnv.chop.edu/) indicate that the described deletion is present in the heterozygous state in the three examined populations. The incidence of the deletion could be particularly high in persons of Asian origin.

TABLE 2
Summary of amplifications at the DPY19L2 locus as
a functon of patient genotype (see FIGS. 2 and 4):

| | | | DPY19L2 gene | | | | | |
|---|---|---|---|---|---|---|---|---|
| PCR | A | b | c | d | e | f | g | h |
| Non-deleted individual | + | + | + | + | + | + | + | − |
| Heterozygous individual | + | + | + | + | + | + | + | + |
| Infertile globozoocephalic | + | − | − | − | − | − | + | + |
| male, DPY19L2 | | | | | | | | |
| homozygous deletion | | | | | | | | |

### Analyse Par CGH Analysis (Comparative Genomic Hybridization)

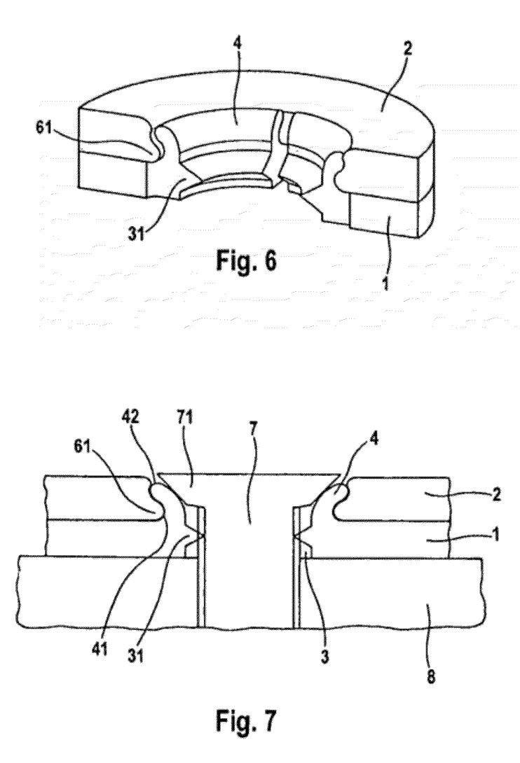

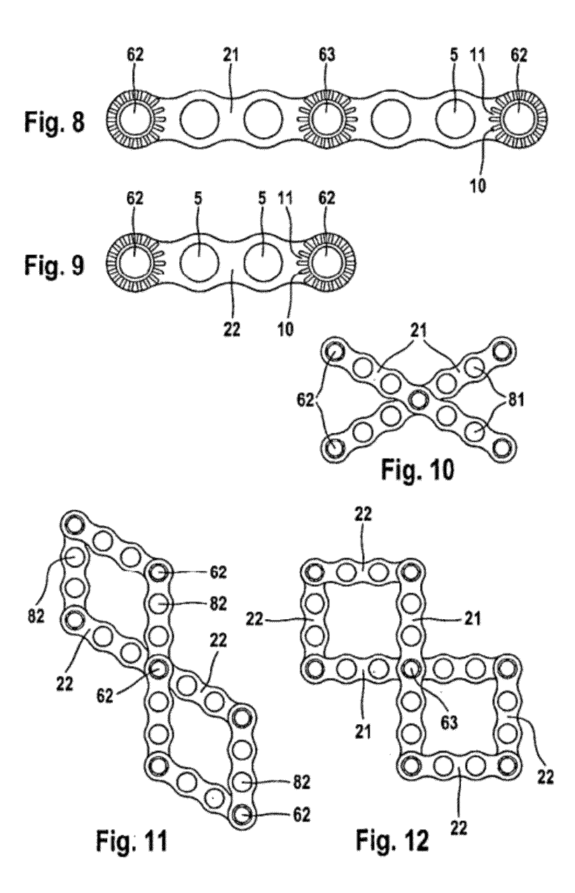

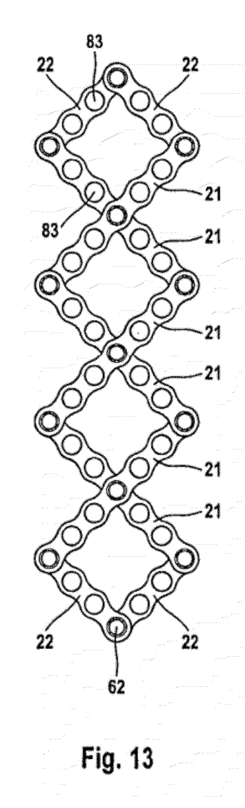

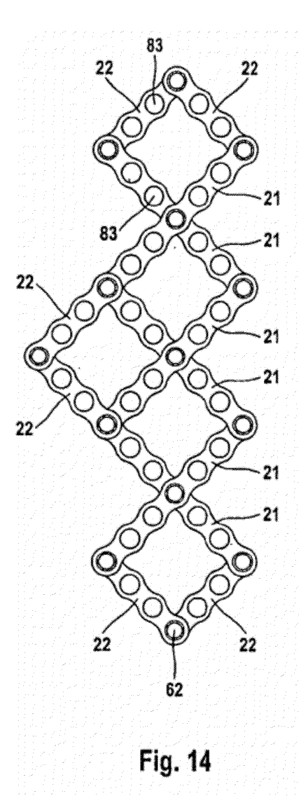

CGH on Agilent 4x180K was carried out in patient P1. The analysis confirmed the presence of a homozygous deletion of DPY19L2 but the analysis did not allow delimiting of the extent of the deletion due to the rarity of probes in this particular region of the Agilent microarray (FIG. 5).

### Discussion

The epidemiological data are given in Table 2 below (pubic data obtained from http://cnv.chop.edu/):

| | | | | Expected |
|---|---|---|---|---|
| | Number of | Number of | Incidence of | incidence of |
| | individuals | heterozygotes | heterozygotes | homozygotes |
| African | 693 | 2 | 1/347 | 1/481 636 |
| European | 1321 | 13 | 1/102 | 1/41 616 |
| Asian | 12 | 1 | 1/12 | 1/576 |
| TOTAL | 2026 | 16 | 1/127 | 1/64 516 |

The expected incidence in Europeans is 1/41 616.

No information is available on the function of DPY19L2. In humans the only data available is given by analysis of cDNA libraries which indicate that the DPY19L2 transcript is chiefly found in the testicles (public data obtained from http://www.cgl.ucsf.edu/Research/genentech/genehub-gepis/enehub-gepis-search.html.showing).

According to its sequence, it is predicted that DPY19L2 is a membrane protein since it contains 11 transmembrane domains.

1. A method for diagnosing infertility in a male human subject, the said method comprising a step comprising detecting, in a biological sample of said subject, the presence or absence of a polynucleotide on chromosome 12, between nucleotide 63,952,693 and nucleotide 64,062,354, of NCBI accession number: NC_{—}000012.11.

2. The method according to claim 1, wherein the homozygous deletion of the said polynucleotide is detected.

3. The method according to claim 1, wherein the heterozygous deletion of the said polynucleotide is detected.

4. The method according to claim 1, wherein the said polynucleotide is the DPY19L2 gene.

5. A method for diagnosing infertility in a male human subject, wherein, in a biological sample of the said subject, no nucleotide sequence identical to SEQ ID NO: 2 is detected, or a nucleotide sequence different from SEQ ID NO: 2 is detected but having at least 95% identity to SEQ ID NO: 2.

6. The method according to claim 5, wherein the nucleotide sequence differing from SEQ ID NO: 2 but having at least 95% identity to SEQ ID NO: 2, does not encode the DPY19L2 protein.

7. The method according to claim 5, wherein prior amplification is performed by means of primers chosen from among the pairs SEQ ID NOS: 3 and 4, SEQ ID NOS: 5 and 6, SEQ ID NOS: 7 and 8, SEQ ID NOS: 9 and 10, SEQ ID NOS: 11 and 12, SEQ ID NOS: 13 and 14, SEQ ID NOS: 15 and 16 and SEQ ID NOS: 17 and 18.

8. The method according to claim 7 wherein negative amplification with at least one of the pairs SEQ ID NOS: 7 and 8, SEQ ID NOS: 9 and 10, SEQ ID NOS: 11 and 12, preferably with the three pairs, is characteristic of homozygous absence of the DPY19L2 gene.

9. The method according to claim 7, wherein positive amplification with at least one of the pairs SEQ ID NOS: 7 and 8, SEQ ID NOS: 9 and 10, SEQ ID NOS: 11 and 12, preferably the three pairs, and positive amplification with the pair SEQ ID NOS: 17 and 18 is characteristic of heterozygous absence of the DPY19L2 gene.

10. The method according to claim 1, wherein the biological sample is chosen from among the leukocytes of peripheral blood and saliva.

11. A vector comprising a polynucleotide encoding a DPY19L2 polypeptide, comprising a polypeptide having a function in the formation of the acrosome of spermatozoa and a sequence having at least 80% identity to SEQ ID No. 1 or a polypeptide of SEQ ID NO: 1, and/or an expression cassette comprising in the direction of transcription a functional promoter, a polynucleotide encoding the said DPY19L2 polypeptide and a terminator sequence, for use thereof in the treatment of a male human subject suffering from DPY19L2-related infertility.

12. A molecule inhibiting the expression or action of a DPY19L2 polypeptide comprising or consisting of a polypeptide having a function in the formation of the acrosome of spermatozoa and a sequence having at least 80% identity to SEQ ID No. 1, in wild-type carrier male subject, the said molecule consisting of an anti-DPY19L2 interfering nucleic acid of DNA or RNA type.

13. The molecule according to claim 12, wherein the polypeptide has the sequence SEQ ID No. 1.

14. The molecule according to claim 12, for use thereof as contraceptive means in wild-type carrier males.

15. A solid substrate of chip type, on which a DPY19L2 polypeptide is fixed comprising or consisting of a polypeptide having a function in the formation of the acrosome of spermatozoa and a sequence having at least 80% identity to SEQ ID No. 1, or a polynucleotide encoding said polypeptide.

16. An antibody specific to the DPY19L2 polypeptide comprising or consisting of a polypeptide having a function in the formation of the acrosome of spermatozoa and a sequence having at least 80% identity to SEQ ID No. 1.

17. A DPY19L2 polypeptide comprising or consisting of a polypeptide having a function in the formation of the acrosome of spermatozoa and a sequence having at least 80% identity to SEQ ID No. 1, for use thereof in a human male subject with a view to restoring functional spermatogenesis.

18. The method according to claim 2, wherein the said polynucleotide is the DPY19L2 gene.

19. The method according to claim 3, wherein the said polynucleotide is the DPY19L2 gene.

20. The method according to claim 1, wherein prior amplification is performed by means of primers chosen from among the pairs SEQ ID NOS: 3 and 4, SEQ ID NOS: 5 and 6, SEQ ID NOS: 7 and 8, SEQ ID NOS: 9 and 10, SEQ ID NOS: 11 and 12, SEQ ID NOS: 13 and 14, SEQ ID NOS: 15 and 16 and SEQ ID NOS: 17 and 18.

21. The method according to claim 5, wherein the biological sample is chosen from among the leukocytes of peripheral blood and saliva.
